(19) United States
(12) Patent Application Publication
(10) Pub. No.: US 2022/0257159 A1
(43) Pub. Date: Aug. 18, 2022

(54) Unitary Plasma Separation Device

(71) Applicant: ViveBio Scientific, LLC, Alpharetta, GA (US)

(72) Inventors: Robert D. Cheeley, Alpharetta, GA (US); Timothy Murray, Roswell, GA (US); Daniel Braun, Carlsbad, CA (US); Brian Weinberg, Carlsbad, CA (US); Ayman Ibrahim, Carlsbad, CA (US)

(21) Appl. No.: 17/596,320

(22) PCT Filed: Jun. 8, 2020
(86) PCT No.: PCT/US2020/036614

§ 371 Date: Dec. 7, 2021

Related U.S. Application Data
(60) Provisional application No. 62/858,591, filed on Jun. 7, 2019.

(51) Int. Cl. A61B 5/15

(52) U.S. Cl. CPC A61B 5/150755; A61B 5/150351; A61B 5/150763

(57) ABSTRACT

Devices and methods are provided that permit efficient and selective separation of liquid biological specimens into at least two constituent components to facilitate subsequent quantitative and qualitative analysis on at least one analyte of interest in at least one of the components. For example, a liquid biological specimen separation device includes a base defining a base surface, a collection housing extending away from the base surface to a collection end, a collection membrane disposed on the collection housing adjacent the collection end, and a cap assembly secured to the collection housing. The cap assembly includes a cap defining an aperture therein configured to allow deposition of a liquid biological specimen therethrough, a separation membrane secured to the cap and extending across the aperture, a cap ring rotatably secured about the collection housing, and a tether coupling the cap to the cap ring.

### CROSS REFERENCE TO RELATED APPLICATIONS

This application is a national stage filing under 35 U.S.C. § 371 of International Application No. PCT/US2020/036614, filed Jun. 8, 2020, which claims the priority benefit of U.S. Provisional Application No. 62/858,591, filed Jun. 7, 2019, each of which is incorporated herein in its entirety by reference.

### TECHNICAL FIELD

The present disclosure generally relates to devices and methods that permit efficient and selective separation of liquid biological specimens (e.g. biological fluids or biological specimen containing suspensions) into at least two constituent components to facilitate subsequent quantitative and qualitative analysis on at least one analyte of interest in at least one of the components.

### BACKGROUND

Biological specimens are often collected for analysis of the levels and concentrations of various analytes contained therein. Although many diagnostics are carried out on biological specimens in their native state, many times the biological specimen must be separated into its constituent components for a variety of reasons. Separating a biological specimen into different constituent parts can maximize the precision, accuracy, and reproducibility of detecting and quantifying analytes of interest within the biological specimen. For example, it is often necessary to filter out solid components from whole blood (e.g., white blood cells, red blood cells, etc.), separate blood serum from whole blood, and separate blood plasma from whole blood, to improve not only the recovery of select analytes from the biological specimen (e.g. viruses, plasma proteins, cytokines, chemokines, immunoglobins, etc.) but also improve the subsequent detection and analysis of those analytes. As one example, red blood cells (erythrocytes) scatter and absorb light and, therefore, can adversely affect diagnostic tests that rely on measurements of either reflected or transmitted light. Removing red blood cells can help obtain the most accurate reading possible.

Traditionally, liquid biological specimens have been separated by centrifugation. For example, blood plasma and serum have been separated from whole blood by centrifuging either before (for plasma) or after (for serum) clotting. However, centrifugation requires electricity and expensive equipment that may not be readily available in a clinical laboratory or out in the field. Further, centrifugation can damage analytes of interest (e.g. nucleic acids such as DNA and RNA).

A number of techniques have been devised to avoid this problem. The techniques generally utilize a filtering device that separates a liquid biological specimen into various components. However, these devices have proven to be unsuitable for a variety of reasons. Therefore, what are needed are improved devices and methods that permit efficient and selective separation of liquid biological specimens into at least two constituent components to facilitate subsequent quantitative and qualitative analysis on at least one analyte of interest in at least one of the components.

### SUMMARY

This disclosure generally provides liquid biological specimen separation devices and methods for separation of liquid biological specimens. In embodiments according to this disclosure, a liquid biological specimen separation device includes a base defining a base surface, a collection housing extending away from the base surface to a collection end, a collection membrane disposed on the collection housing adjacent the collection end, and a cap assembly secured to the collection housing. The cap assembly includes a cap defining an aperture therein configured to allow deposition of a liquid biological specimen therethrough, a separation membrane secured to the cap and extending across the aperture, a cap ring rotatably secured about the collection housing, and a tether coupling the cap to the cap ring.

Devices and methods according to this disclosure may permit efficient and selective separation of liquid biological specimens (e.g. biological fluids or biological specimen containing suspensions) into at least two constituent components to facilitate subsequent quantitative and qualitative analysis on at least one analyte of interest in at least one of the components.

### BRIEF DESCRIPTION OF THE DRAWINGS

Referring now to the drawings, which are meant to be exemplary and not limiting, and wherein like elements are numbered alike. The detailed description is set forth with reference to the accompanying drawings illustrating examples of the disclosure, in which use of the same reference numerals indicates similar or identical items. Certain embodiments of the present disclosure may include elements, components, and/or configurations other than those illustrated in the drawings, and some of the elements, components, and/or configurations illustrated in the drawings may not be present in certain embodiments.

FIGS. 1A to 1D show conceptual views of a liquid biological specimen separation device in a ready configuration.

FIG. 1A is a conceptual perspective view of the liquid biological specimen separation device including a cap assembly including a removable cap disposed on a collection housing in the ready configuration.

FIG. 1B is a conceptual partial cross-sectional view of the liquid biological specimen separation device of FIG. 1A.

FIG. 1C is a conceptual exploded view of the liquid biological specimen separation device of FIG. 1A.

FIG. 1D is a conceptual exploded view of the cap assembly of the liquid biological specimen separation device of FIG. 1A.

FIGS. 2A to 2D show conceptual views of the liquid biological specimen separation device of FIG. 1A transitioning from a ready configuration through an intermediate configuration by rotation of the cap assembly and followed by removal of the cap to transition to a used configuration.

FIG. 2A is a conceptual top view of the liquid biological specimen separation device of FIG. 1A in a ready configuration rotatable to an intermediate configuration.

FIG. 2B is a conceptual top view of the liquid biological specimen separation device of FIG. 2A rotated to the intermediate configuration.

FIG. 2C is a conceptual perspective view of the liquid biological specimen separation device of FIG. 2B moved to a configuration with initiation of cap removal.

FIG. 2D is a conceptual perspective view of the liquid biological specimen separation device of FIG. 2C moved to a configuration with the cap removed from a collection housing and ready for securing to a mount in a used configuration.

FIGS. 3A and 3B show conceptual views of the liquid biological specimen separation device of FIG. 1A transitioned from the ready configuration to a used configuration by rotation of the cap assembly and securement of the cap to a mount.

FIG. 3A is a conceptual perspective view of the liquid biological specimen separation device of FIG. 1A transitioned to the used configuration.

FIG. 3B is a conceptual cross-sectional view of the liquid biological specimen separation device of FIG. 3B in the used configuration

FIGS. 4A to 4D show conceptual views of a liquid biological specimen separation device including a cap including a retaining member in a ready configuration.

FIG. 4A is a conceptual perspective view of the liquid biological specimen separation device including a cap assembly including a removable cap having a reinforced opening disposed on a collection housing in the ready configuration.

FIG. 4B is a conceptual partial cross-sectional view of the liquid biological specimen separation device of FIG. 4A.

FIG. 4C is a conceptual perspective view of a base of the liquid biological specimen separation device of FIG. 4A.

FIG. 4D is a conceptual exploded view of the cap assembly of the liquid biological specimen separation device of FIG. 4A.

FIGS. 5A and 5B show conceptual views of the liquid biological specimen separation device of FIG. 4A transitioned from the ready configuration to a used configuration by rotation of the cap assembly and securement of the cap to a mount.

FIG. 5A is a conceptual perspective view of the liquid biological specimen separation device of FIG. 4A transitioned to the used configuration.

FIG. 5B is a conceptual cross-sectional view of the liquid biological specimen separation device of FIG. 5B in the used configuration.

### DETAILED DESCRIPTION

Devices and methods are provided that permit efficient and selective separation of liquid biological specimens into at least two constituent components to facilitate subsequent quantitative and qualitative analysis on at least one analyte of interest in at least one of the components. The devices and methods fulfill the need for a convenient and simple method for filtering, separating, and/or storing an analyte of interest.

In embodiments according to this disclosure, a liquid biological specimen separation device includes a base defining a base surface, a collection housing extending away from the base surface to a collection end, a collection membrane disposed on the collection housing adjacent the collection end, and a cap assembly secured to the collection housing. The cap assembly includes a cap defining an aperture therein configured to allow deposition of a liquid biological specimen therethrough, a separation membrane secured to the cap and extending across the aperture, a cap ring rotatably secured about the collection housing, and a tether coupling the cap to the cap ring.

Examples of biological specimen suitable for use with devices described herein include whole blood, plasma, urine, saliva, sputum, semen, vaginal lavage, bone marrow, breast milk, and cerebrospinal fluid. One advantage of the present devices is that they can sufficiently preserve analytes of interest.

The devices generally include a base defining a base surface, a collection housing extending away from the base surface to a collection end, a collection membrane disposed on the collection housing adjacent the collection end, and a cap assembly secured to the collection housing. The cap assembly includes a cap defining an aperture therein configured to allow deposition of a liquid biological specimen therethrough, a separation membrane secured to the cap and extending across the aperture, a cap ring rotatably secured about the collection housing, and a tether coupling the cap to the cap ring.

After the specimen is deposited, the cap can be removed from or lifted off the collection housing to space apart the collection membrane and the separation membrane. The collection membrane remains secured in the collection housing, while the separation membrane travels or moves with the cap. The cap can be secured to a different component of the device, for example, a cap mount, in a used configuration of the device. The device may be stored or shipped for subsequent analysis in the used configuration, with the separation membrane and the cap secured spaced apart from the collection membrane and collection housing. Analysis may be performed on the separation membrane retrieved from or retained on the cap or the cap mount, and analysis may be performed on the collection membrane retrieved from or retained on the collection housing. In this way, contamination from dropping the cap or the separation membrane, contact between components of the device and external surfaces or objects, cross-contamination, or transfer of the separated components or fractions of the specimen may be avoided during storage, transport, or otherwise prior to or during analysis.

The devices can be used to trap and filter out solid components from a liquid biological specimen. For example, the devices can include a separation membrane that filters and traps solid components of a whole blood specimen (e.g. red blood cells, white blood cells, erythrocytes), thereby resulting in the collection membrane absorbing cell-free serum, plasma, and plasma proteins.

As used herein, the term “analyte” refers to any micro- or macro-molecules in a biological specimen that are to be detected or analyzed. These include, for example, nucleic acids (e.g. DNA, RNA), polynucleotides, oligonucleotides, proteins, polypeptides, oligopeptides, enzymes, amino acids, receptors, carbohydrates, lipids, whole cells, cellular fragments, any intra- or extra-cellular molecules and fragments, viruses, viral molecules and fragments, bacteria, and the like. In certain embodiments, the analytes are exogenous natural or synthetic compounds such as small molecules like drugs, prodrugs, and metabolites thereof. In certain embodiments, the analytes are nucleic acids such as proviral and/or viral DNA and/or RNA such as, for example, proviral and/or viral nucleic acids from COVID-19, human immunodeficiency virus (HIV), hepatitis B virus (HBV), hepatitis C virus (HCV), influenza, parvovirus B19, or any other human or animal viral pathogen. In certain embodiments, the analytes are viral particles for determining viral load. In certain embodiments, the analytes are biological markers for determining HLA blood types, useful for molecular diagnostic genotyping. In certain embodiments, the analytes are inflammatory biomarkers such as CXCL9/MIG and CXCL10/IP-10. In certain embodiments, the analytes are selected from markers such as free PIGF, dissociated PIGF, sFLT, endogenic(angiogenic), KIM-1, FGF-21, CD274, corticotrophin-releasing factor, defensin, ferritin, lactoferrin, thrombin antithrombin complex, tumor necrosis factor (TNF) alpha receptor 1 (TNFRSF1A; TNFR1; CD120a), insulin-like growth factor-binding protein 4 (IBP4), or sex hormone-binding globulin (SHBG). In certain embodiments, the analytes are micronutrients such as folic acid, homocysteine, retinol binding protein (and/or vitamin A), thyroglobulin, vitamin D, trace metals (e.g. zinc), ferritin, transferrin receptors, methylmalonic acid, holo-transcobalamin, C-reactive protein, and alpha-acid glycoprotein.

“Biological specimen” refers to biologic samples, either in liquid or solid form, having contained therein an analyte of interest. A biological specimen can be, for example, whole blood, plasma, serum, lymph, synovial fluid, bone marrow, cerebrospinal cord fluid, semen, saliva, urine, feces, sputum, vaginal lavage, skin scrapings, hair root cells, or the like of humans or animals; physiological and pathological body liquids such as secretions, excretions, exudates and transudates; any cells or cell components of humans, animals, plants, bacteria, fungi, plasmids, viruses, parasites, or the like that contain analytes of interest, and any combination thereof. In certain embodiments, a biological specimen can be a human body fluid such as whole blood, which can contain analytes of interest such as proviral nucleic acids and/or plasma proteins such as Troponin, monoclonal kappa and lambda free light chains, Cystatin C, and Carbohydrate-Deficient Transferrin (CDT).

“Liquid biological specimen” means a biological fluid or a biological specimen suspended in a fluid medium (e.g. water, saline, etc.). Exemplary liquid biological specimens include human, animal, plant, bacteria, fungi, plasmids, viruses, parasites (e.g. helminthes, protozoas, spirochetes) extracts or suspensions; liquid extracts or homogenates of human or animal body tissues (e.g., bone, liver, kidney, brain); media from DNA or RNA synthesis; mixtures of chemically or biochemically synthesized DNA or RNA; and body fluids/liquids such as whole blood, plasma, serum, synovial fluid, cerebrospinal cord fluid, semen, and saliva.

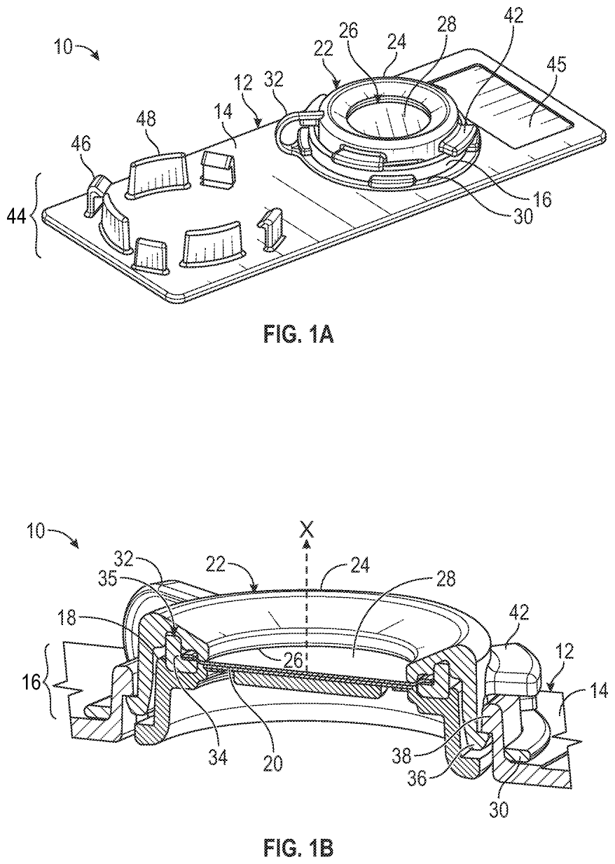

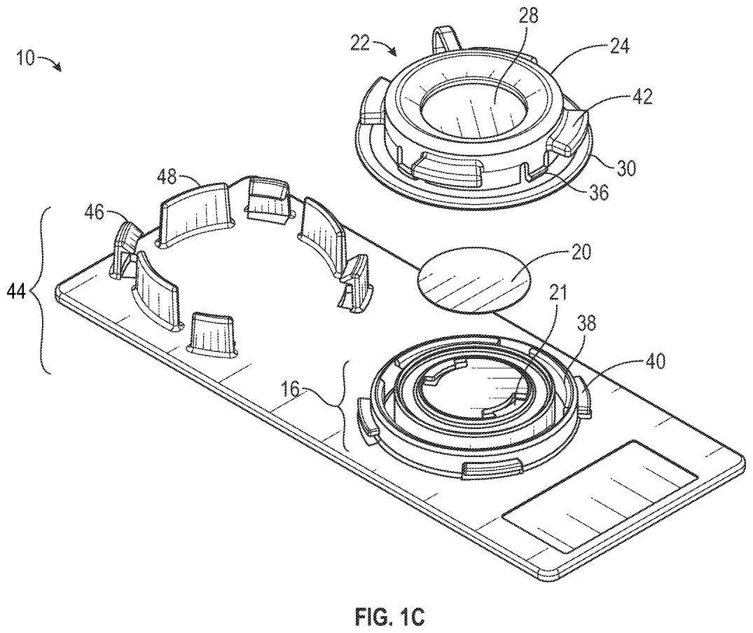

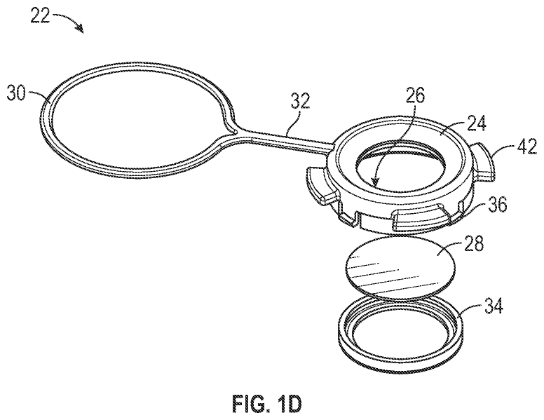

FIGS. 1A to 1D show conceptual views of a liquid biological specimen separation device 10 in a ready configuration. Liquid biological specimen separation device 10 (also referred to in this disclosure as “device 10”) includes a base 12 defining a base surface 14. FIG. 1A is a conceptual perspective view of liquid biological specimen separation device 10 including a cap assembly 22 including a removable cap 24 disposed on a collection housing 16 in the ready configuration. FIG. 1B is a conceptual partial cross-sectional view of liquid biological specimen separation device 10 of FIG. 1A. FIG. 1C is a conceptual exploded view of liquid biological specimen separation device 10 of FIG. 1A.

Collection housing 16 extends away from base surface 14 to a collection end 18. Device 10 further includes a collection membrane 20 disposed on collection housing 16 adjacent collection end 18. The collection membrane 20 generally functions to absorb the fraction of a liquid biological specimen that flows through a separation membrane.

Collection membrane 20 may include any suitable substrate for receiving and collecting a separated component or fraction of a specimen that passes through device 10, for example, through an aperture of a cap and/or a separation membrane. Collection membrane 20 and collection housing 16 can be made of the same material or different materials. Suitable materials include, for example, plastics, polymers, cotton, cellulose, and/or paper. In some embodiments, collection membrane 20 and/or collection housing 16 include filter papers. Filter papers that may be selected for use include cellulose fiber papers manufactured from cotton linters. Cotton linters (i.e., cotton wool) are short fibers that adhere to seeds of a cotton plant after the longer fibers have been pulled from the cotton seed. Filter papers can also include filter papers for blood collection registered by the U.S. Food and Drug Administration as Class II Medical Devices (21 CFR § 862.1675), such as WHATMAN 903, AHLSTROM 142, AHLSTROM 226, AHLSTROM 222, AHLSTROM 238, AHLSTROM 270, ALHSTROM 601, and ESSENTRA. In some embodiments, a majority of the cellulose fibers of a cellulose fiber filter paper may have sizes in the range of about 1-100 microns, 10-50 microns, or 20-25 microns in length.

In some embodiments, collection membrane 20 includes a substantially hydrophobic polyolefin material including a plurality of polypropylene fibers coated with hydrophobic polyethylene. In some embodiments, collection membrane 20 includes microglass fibers.

Collection membrane 20 can include a composition absorbed to a surface thereof, where the composition protects against degradation of an analyte of interest disposed therein. Protection against degradation may include protection against substantial damaging of analytes of interest caused by chemical or biological agents including action of bacteria, free radicals, nucleases, ultraviolet radiation, oxidizing agent, alkylating agents, or acidic agents (e.g., pollutants in the atmosphere). In certain embodiments, the composition absorbed on the collection membrane 20 can include one or more of a weak base, a chelating agent, a protein denaturing agent such as a detergent or surfactant, a nuclease inhibitor, a free radical trap, and an oxygen scavenger element. As used herein, a “weak base” can be a Lewis base which has a pH of about 6 to 10, preferably about pH 8 to 9.5. In a case where the stored analyte of interest is RNA, particularly unstable RNA, the composition may include RNase inhibitors and inactivators, genetic probes, complementary DNA or RNA (or functionally equivalent compounds), proteins and organic moieties that stabilize RNA or prevent its degradation.

Collection housing 16 and/or collection membrane 20 can have any suitable shape such as, for example, a circle, oval, square, rectangle, triangle, hexagonal, or other shapes and surface textures suitable for use in the devices described herein. Collection housing 16 can have a collection membrane aperture for receiving collection membrane 20. Collection housing and collection membrane 20 can also be dimensioned in a manner that facilitates removing collection membrane 20 from collection housing 16. For example, collection housing 16 can include one or more removal apertures that allow a device (e.g. tongs) to selectively pincer and remove collection membrane 20 from collection housing 16. For example, collection membrane 20 may be removed from either the top or the bottom of collection housing 16. In embodiments, automated assemblies or robotic assist systems may be used to disassemble and process large numbers of devices 10, for example, by mechanical or pneumatic means.

Collection housing 16 and/or collection membrane 20 can have any suitable size. In certain embodiments, collection housing 16 and/or collection membrane 20 can have a diameter/width of from about 1 mm to 50 mm, or from 10 mm to 30 mm, inclusive. In some embodiments, collection membrane 20 has a diameter/width of from about 1 mm to about 15 mm.

In some embodiments, collection membrane 20 is reversibly or removably secured to or retained on, in, or adjacent collection end 18 by one or more clips, fasteners, stickers, adhesive, tabs or the like. For example, collection housing 16 may define at least one retaining tab 21 to retain collection membrane 20 on collection end 18. In some embodiments, collection membrane 20 may be removably tucked or secured by tab 21, with a peripheral region of collection membrane 20 held by tab 21.

Device 10 includes a cap assembly 22 including a cap 24. Cap 24 may be disposed on collection housing 16, and is removably securable to collection housing 16. For example, device 10 may be in a ready configuration in which cap 24 is removable secured to collection housing 16, as shown in FIG. 1A. Cap 24 can be made of any suitable flexible, semi-rigid, or rigid material. In some embodiments, cap 24 includes a polymeric material, for example, polyethylene, acrylic, polypropylene, or any other suitable polymer, copolymer, or polymer blend.

FIG. 1D is a conceptual exploded view of cap assembly 22 of liquid biological specimen separation device 10 of FIG. 1A. Cap 24 defines an aperture 26 therein configured to allow deposition of a liquid biological specimen therethrough. Cap assembly 22 includes a separation membrane 28 secured to cap 24 and extending across aperture 26. Separation membrane 28 can be used to separate or fractionate one or more components of the specimen deposited through aperture 26, for example, retaining a fraction on separation membrane 28, while allowing another fraction to passing through separation membrane 28 to collection membrane 20.

Separation membrane 28 is generally made of a material that allows for flow of a liquid biological specimen or a fraction thereof therethrough. Separation membrane 28 can include a plurality of fibers. Separation membrane 28 can be made of a material that has a gradually decreasing pore size (e.g. an asymmetric porous membrane) from a top side (e.g. the side where biological specimens are initially deposited) to a bottom side (the side in contact with or adjacent to collection membrane 20). Separation membrane 28 can also be made of a material that has a uniform pore size throughout. In certain embodiments, flow of a liquid biological specimen deposited on collection membrane 20 through separation membrane 28 is driven by capillary forces (e.g. capillary flow) and/or gravity. In certain embodiments, materials suitable for use in separation membrane 28 are those in which one biological specimen moves faster through the separation membrane than another biological specimen (e.g. blood plasma moves faster than corpuscles).

Suitable materials for use in separation membrane 28 can include, for example, synthetic polymers having fine fiber diameter and fibers made of glass or porous polymers. In certain preferred embodiments, separation membrane 28 are made of a polysulfone polymer material having a porosity that gradually decreases from a top side of the membrane to a bottom side of the membrane so as to filter and trap solid and/or liquid components of a liquid biological specimen deposited on separation membrane 28. Separation membrane materials can include, for example, synthetic or natural polymers such as cellulose mixed esters, polyvinylidene difluoride, polytetrafluoroethylene, polycarbonate, polypropylene, polyester, and polysulfone polymers and matrices (e.g., asymmetric sub-micron polysulfone (BTS) and/or asymmetric super micron polysulfone (MMM) made by Pall Corporation). Separation membrane materials can also include, for example, VIVID GR, VIVID GX, and CYTOSEP 1660. In some embodiments, separation membrane 28 includes a polysulfone polymer material selected from the group consisting of asymmetric sub-micron polysulfone and asymmetric super micron polysulfone. In some embodiments, separation membrane 28 includes a liquid impermeable barrier between two regions of separation membrane 28 formed from collapsed pores in the separation membrane as a result of urging. A person of ordinary skill will readily appreciate that other membranes or filtering materials can be used. In some embodiments, separation membrane 28 is suitable for blood component filtering and serum/plasma separation.

In some embodiments, separation membrane 28 has a porosity of not more than 30%, and preferably not more than 25%. In certain embodiments, separation membrane 28 can be made of polysulfone polymer having a pore size ranging from about 0.1-20 microns and a pore size ratio from about 50:1 to 100:1. Separation membrane 28 may have a porosity that gradually decreases from a first side to a second side so as to filter and trap solid components of a liquid biological specimen deposited on separation membrane 28. Separation membrane 28 may be configured to filter and trap solid components of a biological specimen, the biological specimen being selected from the group consisting of whole blood, plasma, urine, saliva, sputum, semen, vaginal lavages, bone marrow and cerebrospinal fluid. In some embodiments, separation membrane 28 has a pore size ranging from 0.1-20 μm.

The size of separation membrane 28 can be larger than the size of collection membrane 20 to which it is removably disposed upon. For example, the size of a separation membrane 28 may be at least 20%, or at least 30%, or at least 40%, or at least 50% larger than a size of collection membrane 20. Alternatively, the size of separation membrane 28 can be the same or about the same (e.g. within 10% by area) size as that of collection membrane 20. Alternatively, the size of separation membrane 28 can be smaller than the size of collection membrane 20. In certain embodiments, separation membrane 28 has a diameter/width of from about 1 mm to 50 mm, or from 10 mm to 30 mm, inclusive. For example, separation membrane 28 can have a diameter/width of about 10 mm to about 20 mm. In embodiments, a smaller diameter of collection membrane 20 may promote fit of collection membrane 20 in a PCR apparatus, such as in a PCR tube, for subsequent analysis.

Separation membrane 28 can have a shape that is the same shape as collection membrane 20 (e.g. circles). Separation membrane 28 can also have a shape that is different from a shape of collection membrane 20 and, thus, does not align in its entirety with the shape of collection membrane 20 when brought into contact thereto. For example, separation membrane 28 can have an irregular or oblong shape (e.g., a racquet shape with a handle-like extension extending on a lateral side thereof) whereas collection membrane 20 can have a circular shape.

In some embodiments, separation membrane 28 is configured to filter and trap solid components of a whole blood specimen. In some such embodiments, collection membrane 20 is configured to separately filter and trap a plasma fraction or filtrate of the whole blood specimen. In some embodiments, aperture 26, separation membrane 28, and collection membrane 20 are aligned about an axis X extending through a center point of each of aperture 26, separation membrane 28, and collection membrane 20 in the ready configuration, as shown in FIG. 1B.

Cap assembly 22 also includes a cap ring 30 rotatably secured about collection housing 16, and a tether 32 coupling cap 24 to cap ring 30. In some embodiments, tether 32 is U-shaped in the ready configuration. In other embodiments, tether 32 may be V-shaped, angled, zig-zap shaped, or have any other piecewise or completely linear or curved shape.

Cap 24 is removably securable to collection housing 16 at collection end 18 in a ready configuration, as shown in FIGS. 1A to 1C. Separation membrane 28 is disposed adjacent or on collection membrane 20 and ready to receive the liquid biological specimen through aperture 26 in the ready configuration. For example, collection membrane 20 and separation membrane 28 may be substantially aligned in the ready configuration. Separation membrane 28 may be removably or reversibly secured to cap 24 in cap assembly 22 by any suitable clip, fastener, sticker, adhesive, or the like. For example, one or more clips, tabs, fasteners, stickers, or an adhesive layer may secure separation membrane 28 across aperture 26 on cap 24.

In some embodiments, cap assembly 22 further includes a membrane retaining ring 34 about aperture 26. For example, membrane retaining ring may partially, substantially, or completely surround aperture 26. Membrane retaining ring 34 secures separation membrane 28 across aperture 26 of cap 24. For example, membrane retaining ring 34 may hold membrane 28 against a surface of cap 24 such that membrane 28 is stretched, taut, relaxed, or otherwise extended across aperture 26.

Membrane retaining ring 34 may be secured to cap 24 in cap assembly 22 by any suitable clip, sticker, fastener, adhesive, or the like. For example, one or more clips, tabs, stickers, or fasteners, or an adhesive layer may secure membrane retaining ring 34 to cap 24. In some embodiments, cap 24 defines a ring channel 35 about aperture 25, where membrane retaining ring 34 is disposed in ring channel 35, and where separation membrane 28 is between membrane retaining ring 34 and an interior surface of the cap.

Cap 24 may be secured to collection housing 16, for example, to collection end 18 of collection housing 16, by clips, stickers, fasteners, tabs, or any other suitable mechanism. In some examples, cap 24 defines at least one cap tab 36, and collection housing 16 defines at least one mating tab 38. Cap 24 may be removably securable to collection housing 16 by rotatably coupling at least one cap tab 36 and at least one mating tab 38. In some embodiments, at least cap tab 36 includes four cap tabs, and at least mating tab 38 includes four mating tabs, as shown in FIGS. 1A to 1D.

In some embodiments, collection housing 16 defines at least one ring tab 40. Cap ring 30 may be rotatably secured about collection housing 16 by at least one ring tab 40. In some such embodiments, at least one ring tab 40 includes four ring tabs.

In some embodiments, cap 24 defines at least one finger tab 42. At least one finger tab 42 may extend laterally outward from cap 24 and is configured to permit movement of cap 42 relative to collection housing 16. In some embodiments, at least one finger tab 42 includes four finger tabs.

In some embodiments, device 10 further includes a cap mount 44 laterally spaced from collection housing 16 and extending away from base surface 12. Cap 24 and separation membrane 28 may be removably securable to cap mount 44 in a used configuration. Cap 24 and separation membrane 28 are laterally spaced from collection membrane 20 in the used configuration.

Cap ring 30 is rotatably secured about collection housing 16 in the used configuration. Cap ring 30 may be secured about collection housing 16 in both the ready and used configurations, or in other intermediate configurations. Thus, cap ring 30 may retain or secure cap 24 to base 12 of device 10 while permitting cap 24 to be rotated or moved to different configurations or orientations, for example, relative to collection housing 16.

Base 12 generally functions as a supporting surface. Base 12 can also function to secure the base 12, collection membrane 20, separation membranes 28, and/or collection housing 16 together. Base 12 can also serve as a protective enclosure that protects any components that may be contained therein (e.g. collection membrane 20, separation membranes 28, collection housing 16, biological specimens, and/or analytes enclosed by base 12) from outside influences or effects.

Base 12 can assume any dimensions, size, and shape suitable for serving as a support in liquid biological specimen separation device 10. For example, the general shape of base 12 can be round, rectangular, oval, square, trapezoidal, triangular, pentagonal, hexagonal, octagonal, ellipsoid, crescent, curvilinear, egg, quatrefoil, cinquefoil, and the like. Base 12 can have a uniform shape. Base 12 can have a shape that includes one or more lobes/projections extending therefrom. Base 12 can have surfaces that are uniformly flat. Base 12 can be entirely flat. Base 12 can have a three dimensional, freeform structure.

Base 12 can have features that improve handling and/or use of separation device 10. For example, base 12 can include features, projections, tabs, handles, and the like that facilitate gripping, holding, and manipulating separation device 10 (e.g. a tab for use in opening separation device 10 when in a closed configuration).

Base 12 can be made of any suitable material, preferably one that provides sufficient flexibility/stiffness and strength. Base 12 can be made of, for example, suitable plastics materials (e.g. polyethylene, acrylic, polypropylene), paper materials (e.g. cardstock, cardboard, etc.), and the like.

In some embodiments, device 10 further includes an identifier 45 disposed on base 12. For example, identifier 45 may include one or more of alphabetical indicia, numerical indicia, alphanumerical indicia, graphical indicia, symbolic indicia, one-dimensional bar codes, two-dimensional barcodes, quick response (QR) code, radio-frequency identification (RFID) chip or tag, or any other identifier. Identifier 45 may include information may associate device 10 with information such as a particular liquid biological specimen, a source of liquid biological specimen, analysis protocol, constituents, storage duration, storage destination, storage conditions, or the like. Identifier 45 can store or have associated therewith identification information. Identification information can include information specific to a patient associated with a biological specimen stored therein, including personal information (address, name, sex, date of birth, ethnic background, etc.) and/or biometric information (e.g., a fingerprint, a facial image or template). Identifier 45 can also store or have associated therewith contextual information such as time, date, location of testing, and the like. To protect identifier 45 and any information associated therewith, a layer of over laminate or other protective material may additionally be provided over identifier 45.

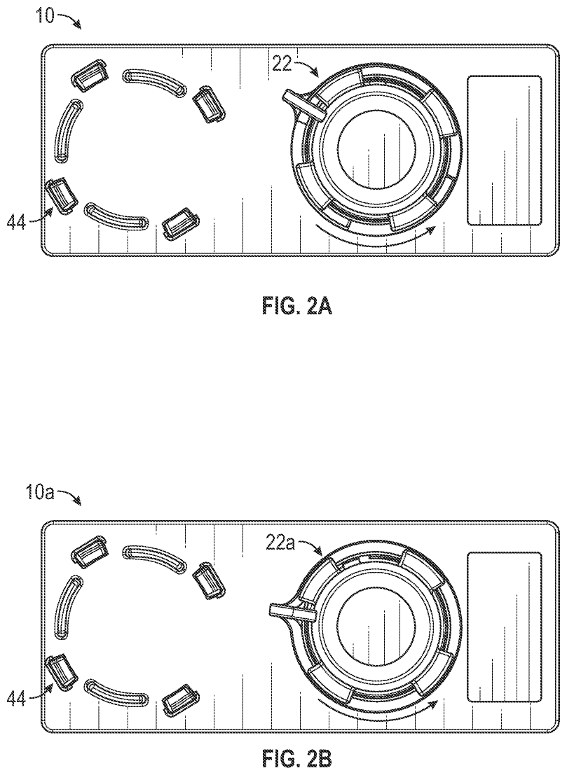

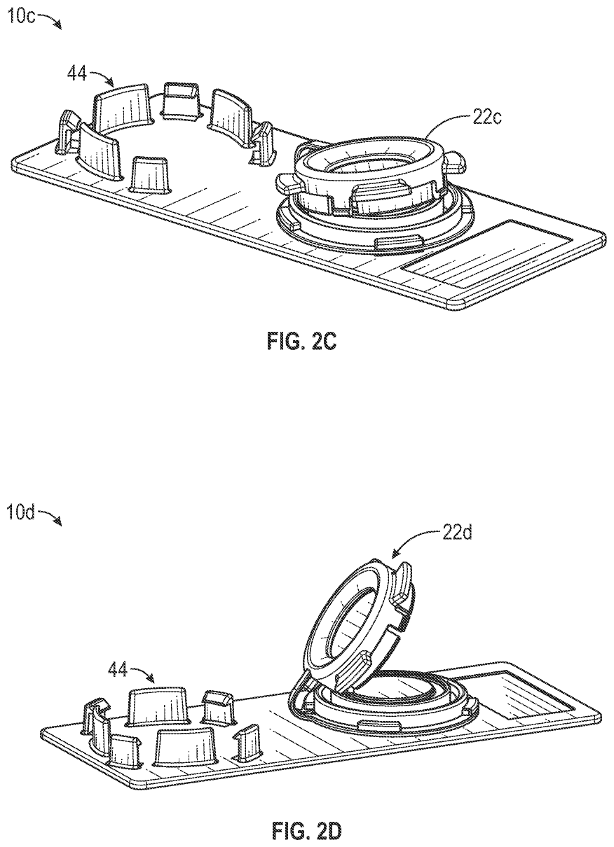

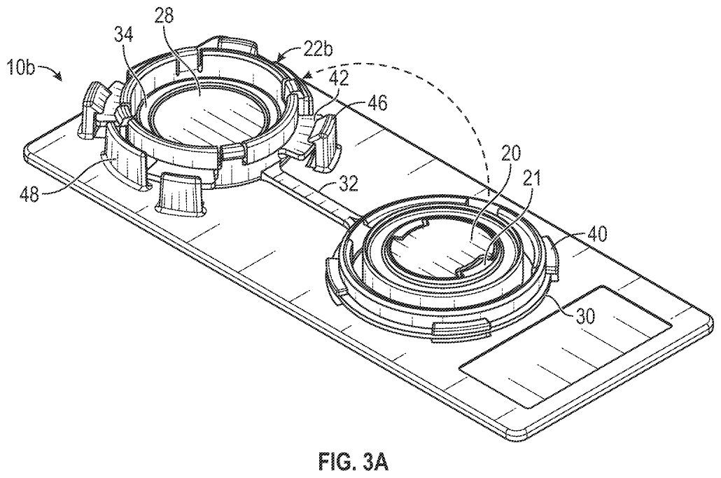

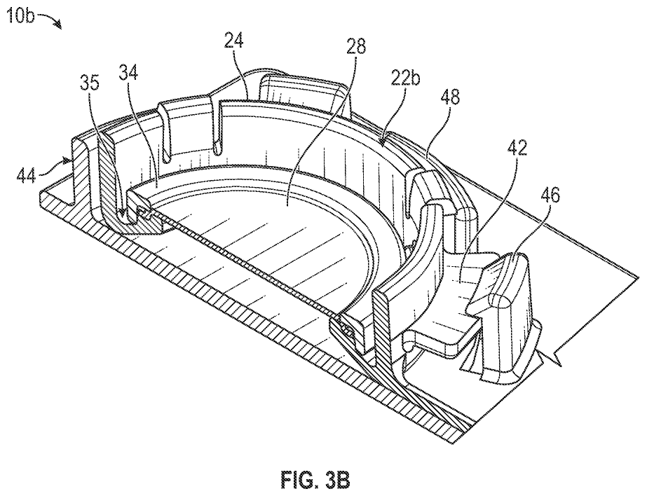

In some embodiments, cap mount 44 includes at least one mount tab 46 for securing cap 24 to cap mount 44. In some such embodiments, at least one mount tab 46 includes four mount tabs. In some embodiments, cap 24 is removably secured to at least one mount tab 46 by at least one finger tab 40. Cap mount 44 may also include additional structure or structures. For example, cap mount 44 may include at least one side wall 48. In some embodiments, side wall 48 extends away from base surface 14 of base 14, and promotes retention of cap 24 on cap mount 44 in the use configuration. In some embodiments, mount tabs 46 and/or side wall 48 may be discrete structures or spaced apart. In other embodiments, cap mount 44 may be integrally formed with mount tabs 46 and/or side wall 48. In some embodiments, side wall 48 may guide one or more portions of cap 24 as cap 24 is moved from the ready configuration to the used configuration, as described with reference to FIGS. 2A, 2B, 3A, and 3D.

FIGS. 2A to 2D show conceptual views of liquid biological specimen separation device 10 of FIG. 1A transitioning from a ready configuration 10 through an intermediate configuration 10a by rotation of cap assembly 22 and followed by removal of cap 24 to transition to a used configuration 10b. FIG. 2A is a conceptual top view of liquid biological specimen separation device 10 of FIG. 1A in ready configuration 10 rotatable to an intermediate configuration 10a. FIG. 2B is a conceptual top view of liquid biological specimen separation device 10 of FIG. 1A rotated to intermediate configuration 10a.

In the ready configuration 10, at least one cap tab 36 may be substantially aligned with at least one mating tab 38 so that cap 24 is secured to and retained on collection end 18 of collection housing 16. As cap assembly 22 is rotated to intermediate configuration 22a associated with intermediate device configuration 10a, cap ring 30 may rotate about collection housing 16, allowing at least one cap tab 36 to slide past at least one mating tab to release from alignment, so that cap 24 is removable from collection housing 16.

Cap 24 can be released from and separated from collection housing 16 of intermediate configuration 10a, and moved to cap mount 44 in the used configuration 10b of device 10, as shown in FIGS. 2C, 2D, 3A and 3D. The configuration 10c and 10d shown in FIGS. 2C and 2D are between the configuration 10a shown in FIG. 2B and the configuration 10b shown in FIGS. 3A and 3B.

FIG. 2C is a conceptual perspective view of liquid biological specimen separation device 10b of FIG. 2B moved to configuration 10c with initiation of cap removal. Cap 24 is partly removed from collection housing 16 to move from cap assembly configuration 22a of FIG. 2B to configuration 22c of FIG. 2C.

FIG. 2D is a conceptual perspective view of liquid biological specimen separation device 10c of FIG. 2C moved to configuration 10d with cap 24 removed from collection housing 16, ready for securing to mount 44 in used configuration 10b.

FIGS. 3A and 3B show conceptual views of liquid biological specimen separation device 10 of FIG. 1A transitioned from ready configuration 10 to a used configuration 10b by rotation of cap assembly 22 and securement of cap 24 to cap mount 44. FIG. 3A is a conceptual perspective view of liquid biological specimen separation device 10 of FIG. 1A transitioned to used configuration 10b. FIG. 3B is a conceptual cross-sectional view of liquid biological specimen separation device 10 of FIG. 3B in used configuration 10b.

As seen in FIGS. 3A and 3B, in the used configuration, separation membrane 28 is retained spaced from collection membrane 20. Device 10 can be stored or shipped in used configuration 10b for subsequent analysis. Analysis may be performed on separation membrane 28 retrieved from or retained on cap 24 or cap mount 44, and/or analysis may be performed on collection membrane 20 retrieved from or retained on collection housing 16. In this way, cross-contamination or transfer of the separated components or fractions of the specimen may be avoided during storage, transport, or otherwise prior to or during analysis.

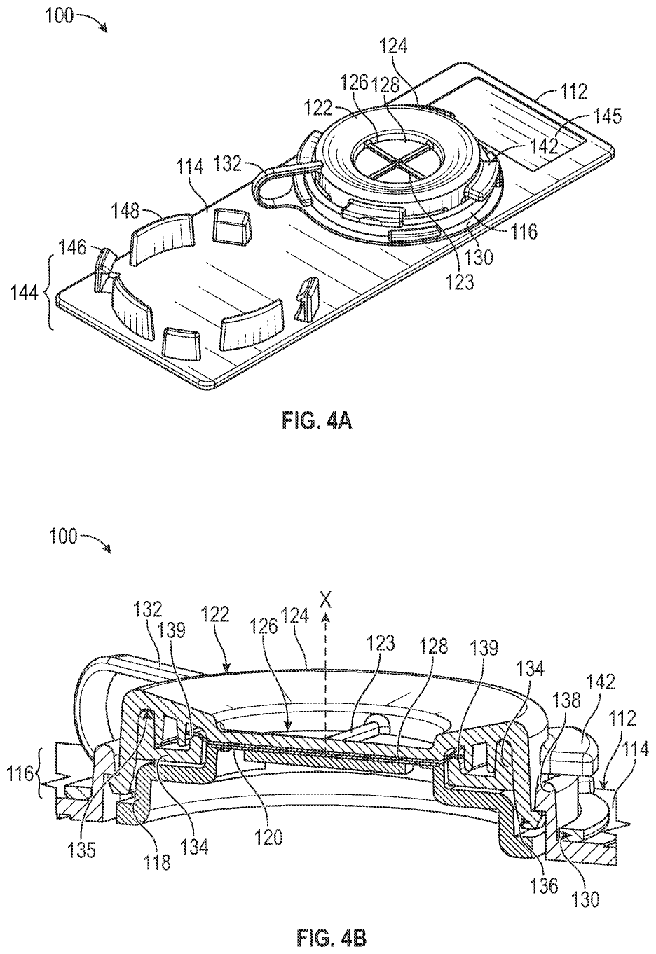

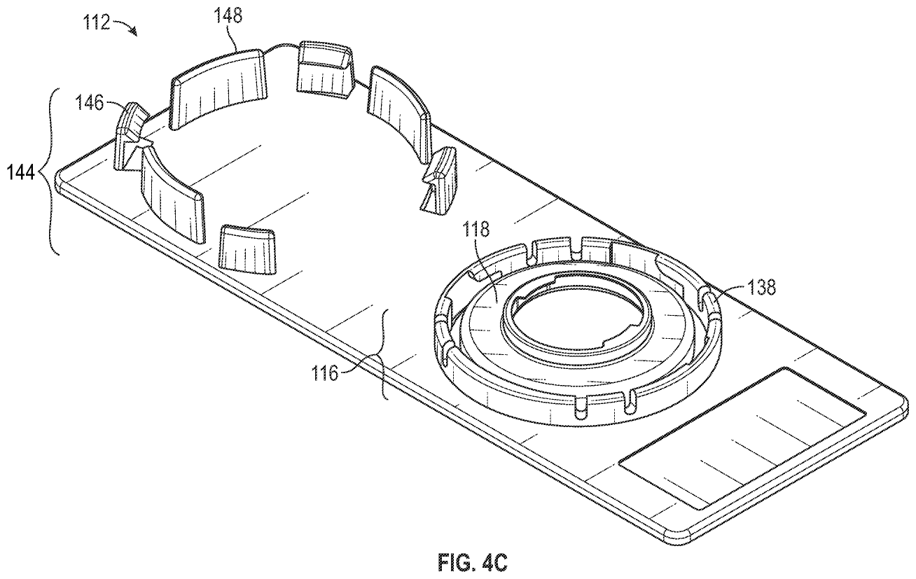

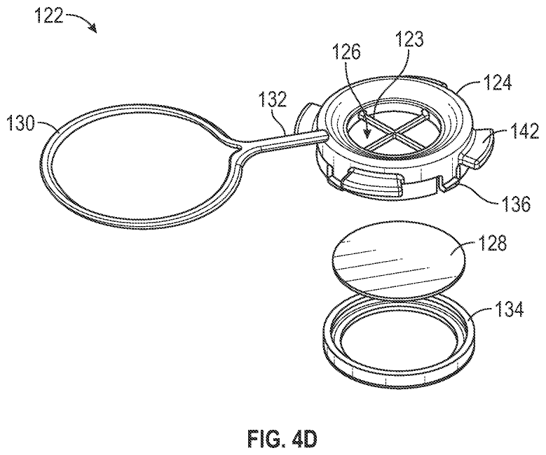

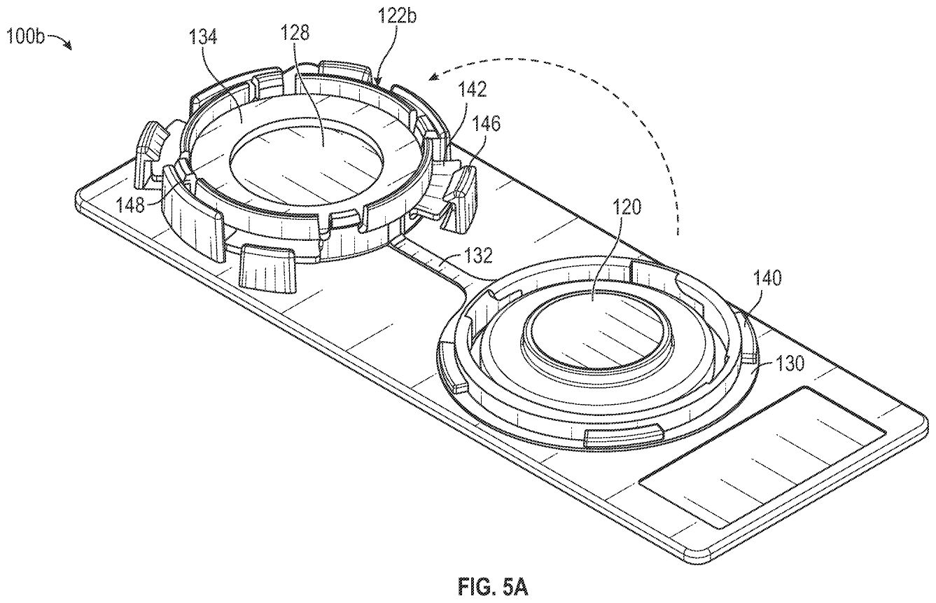

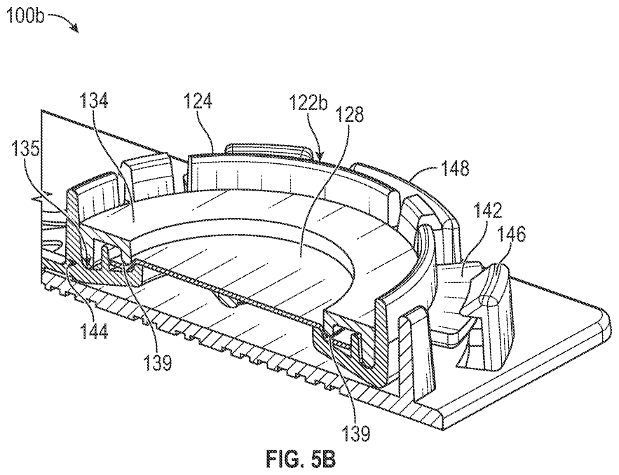

In embodiments, for example, as shown in FIGS. 1A to 3B, aperture 26 of cap 24 is substantially free. For example, no member or structure may extend across aperture 26. In embodiments, as described with reference to FIGS. 4A to 5B, a retaining member may extend across aperture 26. All elements in FIGS. 4A to 5B are illustrated with a three digit reference numeral beginning with 1, and substantially corresponding to similar elements without the leading 1 illustrated in FIGS. 1A to 3B.

FIGS. 4A to 4D show conceptual views of a liquid biological specimen separation device 100 including a removable cap 124 including a retaining member 123 in a ready configuration. Liquid biological specimen separation device 100 is similar to liquid biological specimen separation device 10 of FIGS. 1A to 3B, except for differences described with reference to FIGS. 4A to 5B.

FIG. 4A is a conceptual perspective view of liquid biological specimen separation device 100 including a cap assembly 122 including removable cap 124 having a reinforced opening disposed on a collection housing 144 in the ready configuration. FIG. 4B is a conceptual partial cross-sectional view of the liquid biological specimen separation device 100 of FIG. 4A. FIG. 4C is a conceptual perspective view of base 112 of liquid biological specimen separation device 100 of FIG. 4A. FIG. 4D is a conceptual exploded view of cap assembly 122 of liquid biological specimen separation device 100 of FIG. 4A.

As shown in FIGS. 4A and 4B, cap 124 includes a retaining member 123 extending across an aperture 126 defined by cap 124.

Device 100 includes a base 112 defining a base surface 114, and includes cap assembly 122 including removable cap 124 disposed on collection housing 116 in the ready configuration. Collection housing 116 extends away from base surface 114 to a collection end 118. Device 100 further includes a collection membrane 120 disposed on collection housing 116 adjacent collection end 118. Collection housing 116 may define at least one retaining tab. Cap assembly 122 includes a collection membrane 128 secured to cap 124 and extending across aperture 126.

Cap assembly 122 also includes a cap ring 130 rotatably secured about collection housing 116, and a tether 132 coupling cap 124 to cap ring 130.

In embodiments, retaining member 123 may be unitary or integrated with cap 124, and may be formed or molded as a single or continuous piece with cap 124. In other embodiments, retaining member 123 is formed separately from cap 124, and then secured to cap 124, for example, by welding, adhesive, a clip, a fastener, a slot, or any suitable securing means. Retaining member 123 may be formed of the same material as cap 124, or from a different material than cap 124.

In some embodiments, cap assembly 122 further includes a membrane retaining ring 134 about aperture 126. In embodiments, membrane retaining ring 134 may be the same as or similar to membrane retaining ring 34. In embodiments, membrane retaining ring 134 defines a lip 139, as shown in FIG. 4B. Lip 139 may promote retention of membrane 128 in cap assembly 122, for example, by biasing or securing a peripheral region of membrane 128. In embodiments, lip 139 may extend away from collection housing 116 toward cap 124 such that lip 139 may cause peripheral region or edge of membrane 128 to at least slightly bend or curve, as shown in FIG. 4B.

Retaining ring 134 or lip 139 may be shaped or dimensioned such that only membrane 128 is retained or secured to cap assembly 122, and membrane 120 remains retained or secured to collection housing 116 on relative movement between cap 124 and collection housing 116. In embodiments, collection housing 116 defines at least one tab to promote retention or securing of membrane 120 to collection housing 116. In some embodiments, cap 124 defines a ring channel 135 about aperture 125. Ring channel 135 may define at least one circular rib, for example, a single rib, or two ribs as shown in FIG. 4B, or more ribs. The one or more rib may extend from ring channel 135 and surround aperture 126. The one or more ribs in ring channel 135 may promote engagement of retaining ring with cap 124, while spacing ring 134 or lip 139 at a suitable distance from cap 124. Such spacing may promote contact between ring 139 and end 118 of collection housing 116, which may promote sealing of device 100, or promote secure contact between membranes 120 and 128.

In some examples, cap 124 defines at least one cap tab 136, and collection housing 116 defines at least one mating tab 138. Cap ring 130 may be rotatably secured about collection housing 116 by at least one ring tab 140. In some embodiments, cap 124 defines at least one finger tab 142. In some embodiments, device 100 further includes a cap mount 144 laterally spaced from collection housing 116 and extending away from base surface 112. In embodiments, device 100 further includes an identifier 145 disposed on base 112. In some embodiments, cap mount 144 includes at least one mount tab 146 for securing cap 124 to cap mount 144. Cap mount 144 may include at least one side wall 148. The elements described with reference to device 100 and illustrated in FIGS. 4A to 5B may be the same in structure, shape, configuration, material, construction, and functionality as described with reference to embodiments of device 10 illustrated in FIGS. 1A to 3B. Some differences may be apparent from the disclosure or the figures.

In embodiments, retaining member 123 extends generally flat across aperture 126. In other embodiments, retaining member 123 is biased. For example, retaining member 123 may be curved, bent, or otherwise biased towards housing 116. Such curvature may promote retention or securing of membrane 120 to cap 124, and/or may promote contact between membranes 120 and 128. In embodiments, retaining member 123 is biased toward housing 116, for example, reversibly biased, and may be snapped between two positions. In embodiments, retaining member 123 tends to assume the biased configuration in absence of an external force.

In embodiments, retaining member 123 includes a single member, for example, a single truss or bar, extending across aperture 126. In other embodiments, retaining member 123 includes multiple members. Multiple members of retaining member 123 may contact, touch, meet, or be integrated or continuous, or may be separate members spaced apart from each other. For example, two members may extend in parallel or at a relative lateral angle across aperture 126, without contacting each other. In embodiments, two or more members of retaining member may meet substantially at a center of aperture 126. In some embodiments, as shown in FIGS. 4A to 5B, retaining member 123 includes a cross-hair grid. The cross-hair grid may include a unitary or integrated structure, or may include separate trusses or bars that join substantially at the center of aperture 126.

FIGS. 5A and 5B show conceptual views of the liquid biological specimen separation device of FIG. 4A transitioned from the ready configuration to a used configuration by rotation of the cap assembly and securement of the cap to a mount.

FIG. 5A is a conceptual perspective view of the liquid biological specimen separation device of FIG. 4A transitioned to a used configuration 100b. FIG. 5B is a conceptual cross-sectional view of the liquid biological specimen separation device of FIG. 5B in the used configuration 100b. As seen in FIGS. 5A and 5B, cap 124 may be rotated, removed from collection housing 116, and secured to mount 144, similar to cap 24 described with reference to FIGS. 1A to 3B.

Methods of using a liquid biological specimen separation device are provided. Generally, a liquid biological specimen separation device is used to receive a liquid biological specimen containing an analyte of interest, separate the liquid biological specimen into two components, and store an analyte of interest. The liquid biological specimen separation device is suitable for use as a point-of-care device.

In certain embodiments, the methods include providing a liquid biological specimen separation device and dispensing a liquid biological specimen onto a separation membrane of the device via an aperture in a cover and/or a cap. The liquid biological specimen flows through the separation membrane (e.g. via capillary action, gravity, etc). A first component of the liquid biological specimen is trapped/retained by the separation membrane while a second component of the liquid biological specimen flows through the separation membrane and into the collection membrane, which absorbs the second component. The second component can be dried in the collection membrane, either actively (e.g. via a desiccant or heating) or passively (e.g. air dry), before further processing. Alternatively, the second component can be used for further processing prior to being dried out (e.g. while still wet). The separation membrane and/or the collection membrane having the first and second components respectively can be removed from the device and be exposed to or placed in a reconstitution media to remove/recover analytes of interest therefrom, which can then be analyzed using a suitable technique for the analyte to be studied. In certain embodiments, the methods can include compressing the collection and/or separation membranes to aid in recovering analytes of interest therefrom. In certain embodiments, the methods can include applying reconstitution media to the separation and/or collection membranes to rehydrate analytes of interest contained therein, and compressing the membranes to release the analytes of interest. In certain embodiments, the separation devices and methods separate a liquid biological specimen into at least two constituent components that are subsequently air-dried and stored at ambient temperatures (e.g. without the need for refrigeration or freezing) prior to subsequent quantitative and qualitative analysis on at least one analyte of interest in at least one of the components.

In certain embodiments, the methods include providing a liquid biological specimen separation device. The methods can include adding approximately 25-125 μL of whole blood from either a pipette or directly from a patient's finger-stick or heel-stick onto a top surface of a separation membrane of the device via an aperture in a cover and/or a cap. In embodiments, disposing 25-125 μL or about 70, of whole blood onto a top surface of a separation membrane of the device results in 5-50 μL or about 20 μL of plasma saturating a collection membrane.

The methods can include having a user wait approximately 1, 2, 3, 4, 5, or 10 or more minutes for transfer of plasma through the separation membrane to the collection membrane (e.g. via capillary action, gravity, etc). The methods can include having a user allow the collection membrane to dry (e.g. placing the collection membrane in a designated air drying location) for 1, 2, 3, 4, 5, 6, 12, 24, or more hours. The drying can be accomplished at room temperature by air drying or at controlled temperature. The methods can include placing the whole device or the dried collection membrane in a sealable packaging, which can include a desiccant, for shipment to a laboratory for further analysis. The methods can include the laboratory separating the collection membrane from the separation device. The methods can include the laboratory separating white blood solids from the collection membrane. The methods can include the laboratory suspending the collection membrane in a reconstitution medium.

In certain embodiments, the reconstitution medium is molecular-grade water. In other embodiments, the reconstitution medium includes nuclease-free water or the components of phosphate buffered saline (PBS) or other suitable buffered saline solutions. Optionally, the reconstitution medium includes sodium azide or other antimicrobial agents. The reconstitution medium can also include any number or combinations of available biological preservatives or blood anticoagulants including but not limited to ethylenediaminetetraacetic acid (EDTA), sodium citrate, and heparin. Saline solutions or nuclease-free water can serve as a sterile and neutral medium for the rehydration, re-suspension, and recovery of analyte(s) of interest from the collection and/or separation membranes. When included, antimicrobial agents such as sodium azide prevent microbial growth and subsequent contamination with RNases. When included, biological preservatives such as EDTA, sodium citrate, and heparin serve as anticoagulants and or chelating agents.

The volume of a membrane may or may not expand upon absorption of a liquid biological specimen, and may or may not contract upon drying. However, a liquid saturated membrane can be compressed to release entrained fluid containing an analyte of interest, due to its porosity, by at least about 10%, 20%, 30%, 40%, 50%, 60%, 70%, 75%, 80%, 90%, or more of its saturated volume. Volumetric compression is one convenient technique for releasing analytes of interest, however, other means such as centrifugation or vacuum pressure can alternatively be employed to release analytes of interest from a membrane.

In certain embodiments, the methods can also include an intermediate step of applying a stabilizing composition to the collection membrane and/or the separation membrane to protect analytes of interest against degradation. Depending upon the analytes of interest, the stabilizing composition may include one or more of a weak base, a chelating agent, a protein denaturing agent such as a detergent or surfactant, a nuclease inhibitor, and a free radical trap. Particularly for protection of unstable RNA, the stabilizing composition may include RNase inhibitors and inactivators, genetic probes, complementary DNA or RNA (or functionally equivalent compounds), proteins and organic moieties that stabilize RNA or prevent its degradation.

In embodiments, the time periods for which analytes of interest may be preserved or stored on a collection and/or separation membrane can be for a period of several minutes, hours, days, months, or even greater.

Temperature conditions under which analytes of interest may be preserved or stored on a collection and/or separation membrane are not limited. Typically, analytes of interest are kept at ambient or room temperature, for example, from about 15° C. to about 40° C., preferably from about 15° C. to about 25° C. In some embodiments, the analytes of interest may be kept in a cool environment. For example, in short-term storage, the analytes can be refrigerated at about 2° C. to about 10° C. In yet another example, the analytes may be refrigerated at about 4° C. to about 8° C. In another example, in long-term storage, the analytes can be frozen at about −20° C. to about −80° C. In addition, the membranes may preferably, but not necessarily, be stored in dry or desiccated conditions or under an inert atmosphere.

In certain embodiments, whole blood is dispensed onto a liquid biological specimen separation device. In such embodiments, whole blood or a liquid suspension thereof is deposited onto a separation membrane. The separation membrane absorbs the whole blood. The separation membrane captures some solid components of whole blood (e.g., WBCs, RBCs, platelets, and/or other cellular components) while allowing fluidic and/or other solid whole blood components (e.g. cell-free plasma) to pass through the separation membrane via gravity and/or capillary action. The components of whole blood passing through the separation membrane are absorbed by the collection membrane.

In certain embodiments, cell-free plasma captured on the collection membrane can be removed/recovered from the collection membrane by exposing the collection membrane to a reconstitution media. The recovered cell-free plasma can contain an analyte of interest, for instance, nucleic acids such as DNA and RNA, which can be used for viral load quantitation, genotyping, drug resistance testing, or other suitable analyses. The analytes of interest can be detected or analyzed using analytical and diagnostic methods known in the art.

In some embodiments, a technique for processing or analyzing a specimen may include using a device according to any suitable embodiments of this disclosure. In some embodiments, a technique for separating plasma from whole blood includes securing cap 24 of device 10 to collection end 18 in ready configuration 10, and depositing the liquid biological specimen through aperture 26 and onto separation membrane 28. In some embodiments, the technique further includes, after the depositing, rotating cap 24 about collection housing 16 to release cap 24 and separation membrane 28 from collection end 18, moving cap 24 toward cap mount 44, and securing cap 24 and separation membrane 28 to cap mount 44 in the used configuration 10b. In some embodiments, the liquid biological specimen includes whole blood.

The technique may be used to extract a plasma from whole blood. In some embodiments, the technique may be used such that an amount in a range of 25-125 μL, or 30-100 μL, or 40-90 μL, or 50-80 μL, or 60-75 μL or about 70 μL of whole blood is deposited onto separation membrane 28. In some embodiments, the technique may be used such that an amount in a range of 5-50 μL, or 10-45 μL, or 15-40 μL, or 15-25 μL, or about 20 μL of plasma is recovered in collection membrane 20.

In some embodiments, the technique may be used such that about 70 μL of whole blood is deposited onto separation membrane 28, and about 20 μL of plasma is recovered in collection membrane 20.

Devices and techniques for separation components from samples are described in the disclosure. The following enumerated items describe some aspects according to the disclosure.

Item 1: A liquid biological specimen separation device including:

a base defining a base surface;

a collection housing extending away from the base surface to a collection end;

a collection membrane disposed on the collection housing adjacent the collection end; and

a cap assembly secured to the collection housing, wherein the cap assembly includes:
a cap defining an aperture therein configured to allow deposition of a liquid biological specimen therethrough,
a separation membrane secured to the cap and extending across the aperture,
a cap ring rotatably secured about the collection housing, and
a tether coupling the cap to the cap ring.

Item 2: The device of item 1, wherein the cap is removably securable to the collection housing at the collection end in a ready configuration, wherein the separation membrane is disposed on the collection membrane and ready to receive the liquid biological specimen through the aperture in the ready configuration.

Item 3: The device of item 2, wherein the cap defines at least one cap tab, wherein the collection housing defines at least one mating tab, and wherein the cap is removably securable to the collection housing by rotatably coupling the at least one cap tab and the at least one mating tab.

Item 4: The device of item 3, wherein the at least one cap tab includes four cap tabs, and wherein the at least one mating tab includes four mating tabs.

Item 5: The device of any of items 2 to 4, wherein the tether is U-shaped in the ready configuration.

Item 6: The device of any of items 2 to 5, wherein the aperture, the separation membrane, and the collection membrane are aligned about an axis extending through a center point of each of the aperture, the separation membrane, and the collection membrane in the ready configuration.

Item 7: The device of any of items 1 to 6, wherein the collection housing defines at least one ring tab, wherein the cap ring is rotatably secured about the collection housing by the at least one ring tab.

Item 8: The device of any of items 1 to 7, wherein the cap defines at least one finger tab, wherein the at least one finger tab extends laterally outward from the cap and is configured to permit movement of the cap relative to the collection housing.

Item 9: The device of any of items 1 to 8, further including a cap mount laterally spaced from the collection housing and extending away from the base surface, wherein the cap and separation membrane are removably securable to the cap mount in a used configuration, wherein the cap and the separation membrane are laterally spaced from the collection membrane in the used configuration, and wherein the cap ring is rotatably secured about the collection housing in the used configuration.

Item 10: The device of item 9, wherein the cap mount includes at least one mount tab for securing the cap to the cap mount.

Item 11: The device of item 10, wherein the cap is removably secured to the at least one mount tab by at least one finger tab.

Item 12: The device of any of items 1 to 11, wherein the separation membrane includes a polysulfone polymer material selected from the group consisting of asymmetric sub-micron polysulfone and asymmetric super micron polysulfone

Item 13: The device of any of items 1 to 12, wherein the separation membrane has a porosity that gradually decreases from a first side to a second side so as to filter and trap solid components of a liquid biological specimen deposited on the separation membrane.

Item 14: The device of any of items 1 to 13, wherein the separation membrane is configured to filter and trap solid components of a biological specimen, the biological specimen being selected from the group consisting of whole blood, plasma, urine, saliva, sputum, semen, vaginal lavages, bone marrow and cerebrospinal fluid.

Item 15: The device of any of items 1 to 14, wherein the separation membrane is configured to filter and trap solid components of a whole blood specimen, and wherein the collection membrane is configured to separately filter and trap a plasma fraction or filtrate of the whole blood specimen.

Item 16: The device of any of items 1 to 15, wherein the separation membrane has a pore size ranging from 0.1-20 μm.

Item 17: The device of any of items 1 to 16, wherein the collection membrane includes a substantially hydrophobic polyolefin material including a plurality of polypropylene fibers coated with hydrophobic polyethylene.

Item 18: The device of any of items 1 to 17, wherein one or both of the collection membrane or the separation membrane comprise microglass fibers.

Item 19: The device of any of items 1 to 18, further including an identifier disposed on the base.

Item 20: The device of any of items 1 to 19, wherein the cap assembly further includes a membrane retaining ring about the aperture, wherein the membrane retaining ring secures the separation membrane across the aperture of the cap.

Item 21: The device of item 20, wherein the cap defines a ring channel about the aperture, wherein the membrane retaining ring is disposed in the ring channel, and wherein the separation membrane is between the membrane retaining ring and an interior surface of the cap.

Item 22: The device of any of items 1 to 21, wherein the cap includes a retaining member extending across the aperture.

Item 23: The device of item 22, wherein the retaining member includes a cross-hair grid.

Item 24: The device of item 22 or 23, wherein the retaining member is curved toward the base.

Item 25: A method for separating plasma from whole blood including:

providing a liquid biological specimen separation device including:
a base defining (i) a base surface, (ii) a collection housing extending away from the base surface to a collection end, (iii) a collection membrane disposed on the collection housing adjacent the collection end, and (iv) a cap assembly secured to the collection housing, wherein the cap assembly includes a cap defining an aperture therein configured to allow deposition of a liquid biological specimen therethrough, a separation membrane secured to the cap and extending across the aperture, a cap ring rotatably secured about the collection housing, and a tether coupling the cap to the cap ring

securing the cap of the device to the collection end in a ready configuration; and

depositing the liquid biological specimen through the aperture and onto the separation membrane.

Item 26: The method of item 25, further including, after the depositing:

rotating the cap about the collection housing to release the cap and the separation membrane from the collection end;

moving the cap toward the cap mount; and

securing the cap and the separation membrane to the cap mount in the used configuration.

Item 27: The method of item 25 or 26, wherein the liquid biological specimen includes whole blood.

Item 28: The method of item 27, wherein 70 μL of whole blood is deposited onto the separation membrane.

Item 29: The method of item 28, wherein 20 μL of plasma is recovered in the collection membrane.

The detailed description set forth above is provided to aid those skilled in the art in practicing the invention. However, the invention described and claimed herein is not to be limited in scope by the specific embodiments described above, as these embodiments are presented as mere illustrations of several aspects of the invention. Any combinations and modifications of the described methods and components, and compositions used in the practice of the methods, in addition to those not specifically described, will become apparent to those skilled in the art based on the present disclosure and do not depart from the spirit or scope of the present invention. Such variations, modifications, and combinations are also encompassed by the present disclosure and fall within the scope of the appended claims.

What is claimed is:

1. A liquid biological specimen separation device comprising:
a base defining a base surface;
a collection housing extending away from the base surface to a collection end;
a collection membrane disposed on the collection housing adjacent the collection end; and
a cap assembly secured to the collection housing, wherein the cap assembly comprises:
a cap defining an aperture therein configured to allow deposition of a liquid biological specimen therethrough,
a separation membrane secured to the cap and extending across the aperture,
a cap ring rotatably secured about the collection housing, and
a tether coupling the cap to the cap ring.

2. The device of claim 1, wherein the cap is removably securable to the collection housing at the collection end in a ready configuration, wherein the separation membrane is disposed on the collection membrane and ready to receive the liquid biological specimen through the aperture in the ready configuration.

3. The device of claim 2, wherein the cap defines at least one cap tab, wherein the collection housing defines at least one mating tab, and wherein the cap is removably securable to the collection housing by rotatably coupling the at least one cap tab and the at least one mating tab.

4. The device of claim 3, wherein the at least one cap tab comprises four cap tabs, and wherein the at least one mating tab comprises four mating tabs.

5. The device of claim 2, wherein the tether is U-shaped in the ready configuration.

6. The device of claim 2, wherein the aperture, the separation membrane, and the collection membrane are aligned about an axis extending through a center point of each of the aperture, the separation membrane, and the collection membrane in the ready configuration.

7. The device of claim 1, wherein the collection housing defines at least one ring tab, wherein the cap ring is rotatably secured about the collection housing by the at least one ring tab.

8. The device of claim 1, wherein the cap defines at least one finger tab, wherein the at least one finger tab extends laterally outward from the cap and is configured to permit movement of the cap relative to the collection housing.

9. The device of claim 1, further comprising a cap mount laterally spaced from the collection housing and extending away from the base surface, wherein the cap and separation membrane are removably securable to the cap mount in a used configuration, wherein the cap and the separation membrane are laterally spaced from the collection membrane in the used configuration, and wherein the cap ring is rotatably secured about the collection housing in the used configuration.

10. The device of claim 9, wherein the cap mount comprises at least one mount tab for securing the cap to the cap mount.

11. The device of claim 10, wherein the cap is removably secured to the at least one mount tab by at least one finger tab.

12. The device of claim 1, wherein the separation membrane comprises a polysulfone polymer material selected from the group consisting of asymmetric sub-micron polysulfone and asymmetric super micron polysulfone

13. The device of claim 1, wherein the separation membrane has a porosity that gradually decreases from a first side to a second side so as to filter and trap solid components of a liquid biological specimen deposited on the separation membrane.

14. The device of claim 1, wherein the separation membrane is configured to filter and trap solid components of a biological specimen, the biological specimen being selected from the group consisting of whole blood, plasma, urine, saliva, sputum, semen, vaginal lavages, bone marrow and cerebrospinal fluid.

15. The device of claim 1, wherein the separation membrane is configured to filter and trap solid components of a whole blood specimen, and wherein the collection membrane is configured to separately filter and trap a plasma fraction or filtrate of the whole blood specimen.

16. The device of claim 1, wherein the separation membrane has a pore size ranging from 0.1-20 μm.

17. The device of claim 1, wherein the collection membrane comprises a substantially hydrophobic polyolefin material comprising a plurality of polypropylene fibers coated with hydrophobic polyethylene.

18. The device of claim 1, wherein one or both of the collection membrane or the separation membrane comprise microglass fibers.

19. The device of claim 1, further comprising an identifier disposed on the base.

20. The device of claim 1, wherein the cap assembly further comprises a membrane retaining ring about the aperture, wherein the membrane retaining ring secures the separation membrane across the aperture of the cap.

21. The device of claim 20, wherein the cap defines a ring channel about the aperture, wherein the membrane retaining ring is disposed in the ring channel, and wherein the separation membrane is between the membrane retaining ring and an interior surface of the cap.

22. The device of claim 1, wherein the cap comprises a retaining member extending across the aperture.

23. The device of claim 22, wherein the retaining member comprises a cross-hair grid.

24. The device of claim 22, wherein the retaining member is curved toward the base.

25. A method for separating plasma from whole blood comprising:
providing a liquid biological specimen separation device comprising:
a base defining (i) a base surface, (ii) a collection housing extending away from the base surface to a collection end, (iii) a collection membrane disposed on the collection housing adjacent the collection end, and (iv) a cap assembly secured to the collection housing, wherein the cap assembly comprises a cap defining an aperture therein configured to allow deposition of a liquid biological specimen therethrough, a separation membrane secured to the cap and extending across the aperture, a cap ring rotatably secured about the collection housing, and a tether coupling the cap to the cap ring
securing the cap of the device to the collection end in a ready configuration; and
depositing the liquid biological specimen through the aperture and onto the separation membrane.

26. The method of claim 25, further comprising, after the depositing:
rotating the cap about the collection housing to release the cap and the separation membrane from the collection end;
moving the cap toward the cap mount; and
securing the cap and the separation membrane to the cap mount in the used configuration.

27. The method of claim 25, wherein the liquid biological specimen comprises whole blood.

28. The method of claim 27, wherein 70 μL of whole blood is deposited onto the separation membrane.

29. The method of claim 28, wherein 20 μL of plasma is recovered in the collection membrane.
